(19) United States
(12) Patent Application Publication
(10) Pub. No.: US 2022/0233734 A1
(43) Pub. Date: Jul. 28, 2022

(54) DISINFECTION AND STERILIZATION APPARATUS AND METHOD FOR REFRIGERATED CONTAINER TRANSPORTATION SYSTEM

(71) Applicant: Guangdong University of Petrochemical Technology, Maoming City (CN)

(72) Inventors: Gengyu CAO, Maoming City (CN); Changhao WANG, Maoming City (CN); Qun WANG, Maoming City (CN)

(21) Appl. No.: 17/586,887

(22) Filed: Jan. 28, 2022

(30) Foreign Application Priority Data
Jan. 28, 2021 (CN) 202110118349.5

(51) Int. Cl. A61L 2/20; A61L 2/24; A23L 3/3409; A23L 3/00; A23L 3/36

(52) U.S. Cl. CPC A61L 2/202; A61L 2/24; A23L 3/3409; A23L 3/001; A23V 2002/00; A23L 3/36; A61L 2202/14; A61L 2202/121; A61L 2202/122; A23L 3/003

(57) ABSTRACT

Disclosed are a disinfection and sterilization apparatus and method for a refrigerated container transportation system. The disinfection and sterilization apparatus comprises a to-be-treated waiting area, a disinfection and sterilization system, a treatment waiting area and a central control system, wherein a first prompter is arranged in the to-be-treated waiting area; the disinfection and sterilization system comprises a disinfection and sterilization bin, a vacuumizing device and an ozone supply device; a disinfection and sterilization bin inlet and a disinfection and sterilization bin outlet are respectively formed in the left side and the right side of the disinfection and sterilization bin; the top of the disinfection and sterilization bin is provided with a detaching and hanging device for detaching or hanging a refrigerated container and a transportation vehicle; a second prompter is arranged in the treatment waiting area; and the central control system is connected with the first prompter, the disinfection and sterilization bin inlet, the disinfection and sterilization bin outlet, the detaching and hanging device and the second prompter through data transmission lines. According to the disinfection and sterilization apparatus and method, a new coronavirus propagation path of cold-chain food in the refrigerated container can be effectively, and particularly, adverse effects of disinfection and sterilization apparatuses on parts such as engines and tires of transportation vehicles in the vacuumizing and ozone injection processes can be avoided.

### TECHNICAL FIELD

The present disclosure relates to the technical field of cold-chain transportation disinfection and sterilization, in particular to a disinfection and sterilization apparatus and method for a refrigerated container transportation system.

### BACKGROUND ART

For prevention and control of major infectious diseases, public health means are more important than medical means. The novel coronavirus also has a certain period of survival time outside the human body, and can be adsorbed on the surface of an object to cause contact transmission. Many cases show that positive novel coronavirus nucleic acids are detected on some cold-chain food and/or cold-chain food packages and/or cold-chain food transportation tools. Cold chains can preserve food and also serve as routes for virus transmission, and once cold-chain food and/or cold-chain food packages and/or cold-chain food transportation tools are contaminated with novel coronavirus during production, processing and circulation, the low-temperature and high-humidity environment of traditional cold chains is conducive to long-term vigor of the virus, which can cause epidemic situations. How to scientifically and effectively cut off the transmission paths of novel coronavirus on cold-chain food, cold-chain food packages and cold-chain food transportation tools and strengthen the supervision of cold-chain food and all links in circulation is also important.

Existing disinfection and sterilization devices easily cause adverse effects on parts such as engines and tires of transportation vehicles in the refrigerated container transportation system if directly disinfecting and sterilizing the refrigerated container transportation system.

Therefore, there is a need for an effective disinfection and sterilization device and method for cold-chain logistics transportation, which cut off the transmission paths of novel coronavirus on cold-chain food and/or cold-chain food packages and/or cold-chain food transportation tools without destroying the parts such as engines and tires of transportation vehicles.

### SUMMARY

A first technical problem to be solved by the present disclosure is to provide a disinfection and sterilization apparatus for a refrigerated container transportation system. The disinfection and sterilization apparatus is simple in structure and easy to implement, can effectively cut off the transmission paths of novel coronavirus on the surfaces of cold-chain food and cold-chain food packages in cold-chain food transportation tools, avoids contact between workers and cold-chain articles, and avoids contact infection, and furthermore, adverse effects of disinfection and sterilization apparatuses on parts such as engines and tires of transportation vehicles in vacuumizing and ozone injection processes can be avoided.

A second technical problem to be solved by the present disclosure is to provide a disinfection and sterilization method for a refrigerated container transportation system.

In the present disclosure, the term “refrigerated container transport system” is composed of a refrigerated container and a transportation vehicle; and the refrigerated container is carried by the transportation vehicle.

In order to solve the first technical problem, the present disclosure adopts the following technical scheme:

The disinfection and sterilization apparatus for a refrigerated container transportation system comprises

a to-be-treated waiting area, a disinfection and sterilization system, a treatment waiting area and a central control system, wherein

the to-be-treated waiting area, the disinfection and sterilization system and the treatment waiting area are sequentially arranged from left to right;

a first prompter is arranged in the to-be-treated waiting area; the to-be-treated waiting area is a waiting area for the to-be-disinfected and to-be-sterilized refrigerated container transportation vehicle;

the disinfection and sterilization system comprises a disinfection and sterilization bin, a vacuumizing device and an ozone supply device; the disinfection and sterilization bin is an operation area for disinfecting and sterilizing the refrigerated container, and is a closable space capable of accommodating the refrigerated container transportation vehicle to get in and out; a disinfection and sterilization bin inlet and a disinfection and sterilization bin outlet are respectively formed in the left side and the right side of the disinfection and sterilization bin; the disinfection and sterilization bin communicates with the vacuumizing device through a pipeline, and the vacuumizing device can vacuumize the interior of the disinfection and sterilization bin; and the disinfection and sterilization bin communicates with the ozone supply device through a pipeline, and the ozone supply device can fill the disinfection and sterilization bin with ozone;

the disinfection and sterilization bin is provided with a detaching and hanging device for detaching or hanging a refrigerated container and a transportation vehicle;

a second prompter is arranged in the treatment waiting area, the treatment waiting area is a waiting area for the transportation vehicle when the refrigerated container performs disinfection and sterilization operation in the disinfection and sterilization bin; and

the central control system is connected with the first prompter, the disinfection and sterilization bin inlet, the disinfection and sterilization bin outlet, the detaching and hanging device and the second prompter through data transmission lines, and the central control system is used for controlling the operation and the closing of the first prompter, the disinfection and sterilization bin inlet, the disinfection and sterilization bin outlet, the detaching and hanging device and the second prompter.

In an embodiment, the disinfection and sterilization apparatus is further provided with a coding device; and the operation and the closing of the coding system are controlled by the central control system.

In an embodiment, the disinfection and sterilization apparatus is further internally provided with a refrigerating device, and the operation and the closing of the refrigerating system are controlled by the central control system.

In an embodiment, the vacuumizing device comprises a vacuumizing pump.

In an embodiment, the ozone supply device comprises an ozone generator and an ozone pump.

In an embodiment, the disinfection and sterilization bin inlet and the disinfection and sterilization bin outlet are the same port, and the to-be-treated waiting area and the treatment waiting area are the same area.

In an embodiment, a vibration device is arranged on the detaching and hanging device, so that the detaching and hanging device has a vibration function.

In order to solve the second technical problem, the present disclosure adopts the following scheme:

A disinfection and sterilization method for a refrigerated container transportation system comprises the following steps:

S1, enabling a refrigerated container transportation vehicle to wait in a to-be-treated waiting area;

S2, sending an instruction to a first prompter to prompt the refrigerated container transportation vehicle to enter a disinfection and sterilization bin by a central control system;

S3, enabling the refrigerated container transportation vehicle to enter the disinfection and sterilization bin through the disinfection and sterilization bin inlet and stop stably;

S4, controlling the detaching and hanging device to operate by the central control system, so that the refrigerated container is lifted to be detached from the transportation vehicle;

S5, controlling a second prompter to send an instruction to prompt the transportation vehicle to leave the disinfection and sterilization bin by the central control system;

S6, opening a door of the refrigeration container and/or a ventilation opening of the refrigeration container to guarantee the vacuumizing effect and the ozone injection effect;

S7, closing the disinfection and sterilization bin inlet and the disinfection and sterilization bin outlet, so that the disinfection and sterilization bin is closed;

S8, starting a vacuumizing system to vacuumize the disinfection and sterilization bin;

S9, starting an ozone supply device to fill ozone into the disinfection and sterilization bin, so that cold-chain materials in the refrigerated container, cold-chain material packages and gaps of the refrigerated container are filled with ozone;

S8, enabling the pressure of the injected ozone to be 0.8 to 1.2 atmospheric pressures;

S11, maintaining the pressure of the ozone in the disinfection and sterilization bin for 1-3600 seconds; closing the door of the refrigeration container and/or the ventilation opening of the refrigeration container;

S12, controlling to open the disinfection and sterilization bin outlet by a central controller, and meanwhile, sending an instruction to prompt the transportation vehicle to return to the disinfection and sterilization bin by the second prompter;

S13, enabling the transportation vehicle to stop stably, and controlling the detaching and hanging device to operate by the central controller, so that the container descends to the transportation vehicle; and

S14, sending an instruction to the second prompter to prompt the refrigerated container transportation vehicle to drive out of the disinfection and sterilization bin by the central controller.

In an embodiment, in step S14, before the refrigerated container transportation vehicle is driven out of the disinfection and sterilization bin, a coding device is used for coding and recording on the refrigerated container transportation vehicle.

Any range recorded herein includes the endpoints, any numerical value between the endpoints and any subranges subsumed by the endpoints or any numerical value between the endpoints.

If not specified, each raw material in the present disclosure is commercially available, and the equipment used in the present disclosure may be used by using conventional equipment in the art or by reference to the prior art in the art.

Compared with the prior art, the present disclosure has the following beneficial effects:

firstly, according to the disinfection and sterilization apparatus and the disinfection and sterilization method, novel coronavirus on cold-chain food, cold-chain food packages and cold-chain food transportation tools can be effectively killed;

secondly, according to the disinfection and sterilization apparatus and the disinfection and sterilization method, the novel coronavirus can be killed in the whole container without loading and unloading goods, and the goods are disinfected, sterilized and left so that the efficiency is high;

thirdly, the door of the disinfected and sterilized container is sealed and coded, so that the traceability is realized;

fourthly, adverse effects of vacuumizing and ozone injection on trailer engines and tires in the disinfection and sterilization process are avoided;

fifthly, when the detaching and hanging device runs to enable the container to descend and the trailer to be hung, the container is vibrated, and when the trailer is started to run, the container is vibrated, so that goods in the container are loosened, and ozone is favorably diffused in all spaces and gaps; and a vibration function is added, all spaces and gaps are filled with ozone, so that disinfection and sterilization are facilitated; and

sixthly, after the ozone used in the apparatus is reacted, high-concentration oxygen can keep the freshness of cold-chain materials.

In a word, the disinfection and sterilization apparatus and the disinfection and sterilization method can effectively cut off the transmission paths of novel coronavirus on cold-chain food, cold-chain food packages and cold-chain food transportation tools.

### BRIEF DESCRIPTION OF THE DRAWINGS

The following further describes specific embodiments of the present disclosure with reference to the following attached figures.

FIG. 1 is a schematic state diagram of a refrigerated container transportation system when in a to-be-treated waiting area of the present disclosure;

FIG. 2 is a schematic state diagram of a refrigerated container transportation system when entering a disinfection and sterilization bin of the present disclosure;

FIG 3 is a schematic diagram of the disinfection and sterilization state of a refrigerated container in a disinfection and sterilization bin;

FIG. 4 is a schematic state diagram of a refrigerated container when being ready to leave a disinfection and sterilization bin after disinfection and sterilization; and

FIG. 5 is a control block diagram of a central control system of the present disclosure.

### DETAILED DESCRIPTION OF THE EMBODIMENTS

In order to describe the present disclosure more clearly, the present disclosure is further described in detail in conjunction with the attached figures. Those skilled in the art will appreciate that what is specifically described below is illustrative and not restrictive, and should not limit the scope of the present disclosure.

It should be noted that when one element is referred to as being “fixed” or “arranged” on the other element, it can be directly on the other element or indirectly on the other element. When one element is referred to as being “connected” to the other element, it may be directly connected to the other element or indirectly connected to the other element.

The indicative direction or position relations of the terms such as “length”, “width”, “upper”, “lower”, “front”, “back”, “left”, “right”, “vertical”, “horizontal”, “top”, “bottom”, “inside” and “outside” are direction or position relations illustrated based on the accompanying diagrams, just for facilitating the description of the present disclosure and simplifying the description, but not for indicating or hinting that the indicated device or element must be in a specific direction and is constructed and operated in the specific direction, the terms cannot be understood as the restriction of the present disclosure.

In addition, the terms “first” and “second” are merely intended for a purpose of description, and shall not be understood as an indication or implication of relative importance or implicit indication of the number of indicated technical features. Therefore, a feature limited by “first” or “second” may include one or more features explicitly or implicitly. In the description of the present disclosure, the meaning of “a plurality of” means two or more unless expressly specifically defined otherwise.

### Embodiment I

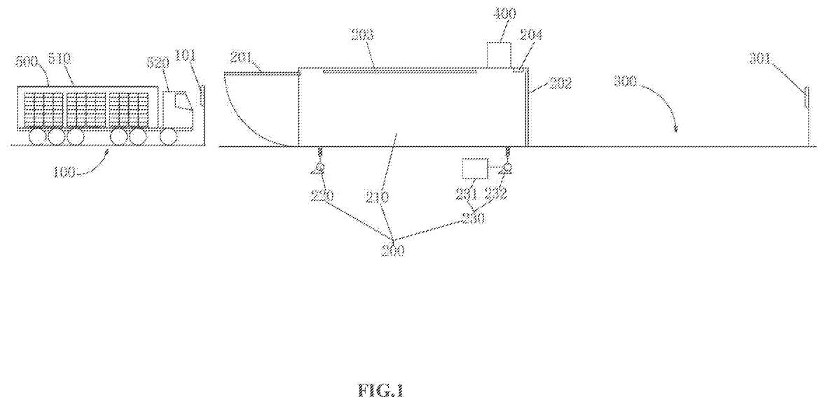

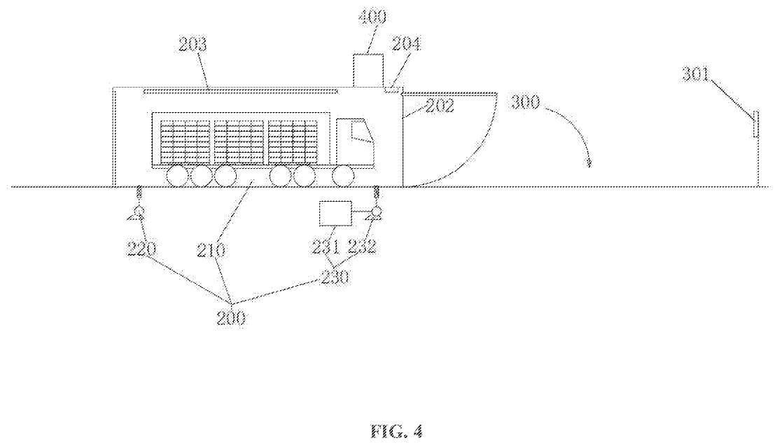

As shown in FIG. 1 to FIG. 4, the disinfection and sterilization apparatus for a refrigerated container transportation system comprises

a to-be-treated waning area 100, a disinfection and sterilization system 200, a treatment waiting area 300 and a central control system 400, wherein

the to-be-treated waiting area 100, the disinfection and sterilization system 200 and the treatment waiting area 300 are sequentially arranged from left to right;

a first prompter 101 is arranged in the to-be-treated waiting area 100; the to-be-treated waiting area 100 is a waiting area for the to-be-disinfected and to-be-sterilized refrigerated container transportation vehicle 500; the refrigerated container transportation vehicle 500 is composed of a refrigerated container 510 and a transportation vehicle 520;

the disinfection and sterilization system 200 comprises a disinfection and sterilization bin 210, a vacuumizing device 220 and an ozone supply device 230; the disinfection and sterilization bin 210 is an operation area for disinfecting and sterilizing the refrigerated container 510, and is a closable space capable of accommodating the refrigerated container transportation vehicle 500 to get in and out; a disinfection and sterilization bin inlet 201 and a disinfection and sterilization bin outlet 202 are respectively formed in the left side and the right side of the disinfection and sterilization bin 210, the disinfection and sterilization bin 200 communicates with the vacuumizing device 220 through a pipeline, and the vacuumizing device 220 can vacuumize the interior of the disinfection raid sterilization bin 200, and the disinfection and sterilization bin 210 communicates with the ozone supply device 230 through a pipeline, and the ozone supply device 230 can fill the disinfection and sterilization bin 210 with ozone;

the top of the disinfection and sterilization bin 210 is provided with a detaching and hanging device 203 for detaching or hanging a refrigerated container 510 and a transportation vehicle 520; it can be understood that, after the detaching and hanging device is used to lift the refrigerated container 510 to detach off the surface of the transportation vehicle 520, the refrigerated container 510 is hung in the disinfection and sterilization bin 210 as the transportation vehicle 520 leaves, so that a blind zone in ozone disinfection and sterilization caused by contact of the refrigerated container 510 with the bottom of the disinfection and sterilization bin is avoided; meanwhile, the transportation vehicle 520 leaves the disinfection and sterilization bin 210 and stops at the treatment waiting area 300, and because the transportation vehicle is not in the disinfection and sterilization bin in the disinfection and sterilization process, the adverse effects of vacuumizing and ozone injection on parts such as engines and tires of the transportation vehicle in the disinfection and sterilization bin in the disinfection and sterilization process are avoided; and as a changed embodiment, it is also within the scope of the present disclosure that the detaching and hanging device 203 can be arranged on the ground to detach or hang the refrigerated container 510 and the transportation vehicle 520 in a supported manner;

a second prompter 301 is arranged in the treatment waiting area 300; the treatment waiting area 300 is a waiting area for the transportation vehicle 520 when the refrigerated container 510 performs disinfection and sterilization operation in the disinfection and sterilization bin; and

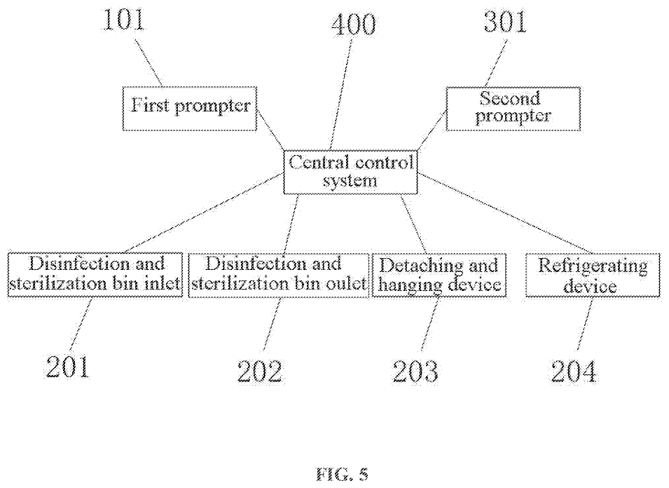

as shown in FIG. 5, the central control system 400 is connected with the first prompter 101, the disinfection and sterilization bin inlet 201, the disinfection and sterilization bin outlet 202, the detaching and hanging device 203 and the second prompter 301 through data transmission lines, and the central control system 400 is used for controlling the operation and the closing of the first prompter 101, the disinfection and sterilization bin inlet 201, the disinfection and sterilization bin outlet 202, the detaching and hanging device 203 and the second prompter 301.

As a preferable embodiment, the disinfection and sterilization apparatus is further provided with a coding device (unshown in the figures); and the operation and the closing of the coding system are controlled by the central control system.

As a preferable embodiment, the disinfection find sterilization apparatus 210 is further internally provided with a refrigerating device 204; and the operation and the closing of the refrigerating system are controlled by the central control system.

As a preferable embodiment, the vacuumizing device 220 comprises a vacuumizing pump.

As a preferable embodiment, the ozone supply device 230 comprises an ozone generator 231 and an ozone pump 232.

As a preferable embodiment, a vibration device is arranged on the detaching and hanging device 203, so that the detaching and hanging device has a vibration function.

As a preferable embodiment, the disinfection and sterilization bin inlet 201 and the disinfection and sterilization bin outlet 202 are the same port, and the to-be-treated waiting area 100 and the treatment waiting area 300 are the same area. It can be understood that the disinfection and sterilization bin provided with a disinfection and sterilization bin inlet and a disinfection and sterilization bin outlet is a more convenient choice for use; as a variation, the refrigerated container transportation vehicle can enter or exit through only one port, and only one to-be-treated waiting area can be arranged to achieve the disinfection and sterilization process of the present disclosure; and however, this variation clearly makes the disinfection and sterilization process less smooth.

### Embodiment II

A disinfection and sterilization method for a refrigerated container transportation system comprises the following steps:

S1, enabling a refrigerated container transportation vehicle to wait in a to-be-treated waiting area, as shown in FIG. 1;

S2, sending an instruction to a first prompter to prompt the refrigerated container transportation vehicle to enter a disinfection and sterilization bin by a central control system;

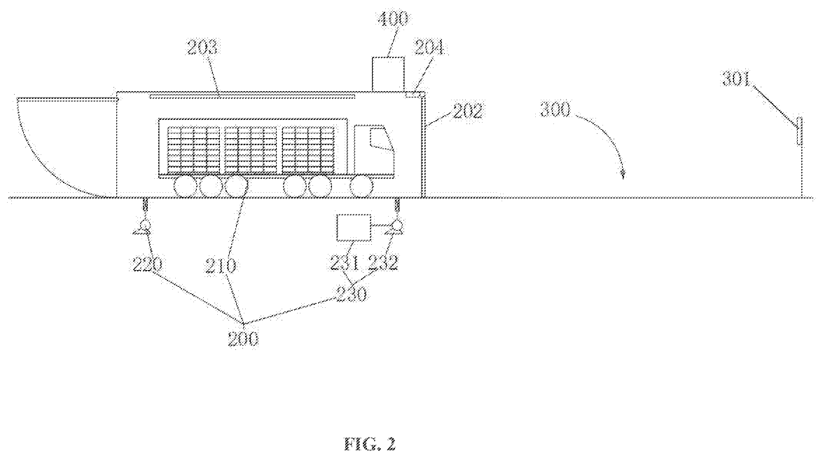

S3, enabling the refrigerated container transportation vehicle to enter the disinfection and sterilization bin through the disinfection and sterilization bin inlet and stop stably, as shown in FIG. 2,

S4, controlling the detaching and hanging device to operate by the central control system, so that the refrigerated container is lifted to be detached from the transportation vehicle;

S5, controlling a second prompter to send an instruction to prompt the transportation vehicle to leave the disinfection and sterilization bin by the central control system;

S6, opening a door of the refrigeration container and/or a ventilation opening of the refrigeration container to guarantee the vacuumizing effect and the ozone injection effect;

S7, closing the disinfection and sterilization bin inlet and the disinfection and sterilization bin outlet, so that the disinfection and sterilization bin is closed;

S8, starting a vacuumizing system to vacuumize the disinfection and sterilization bin;

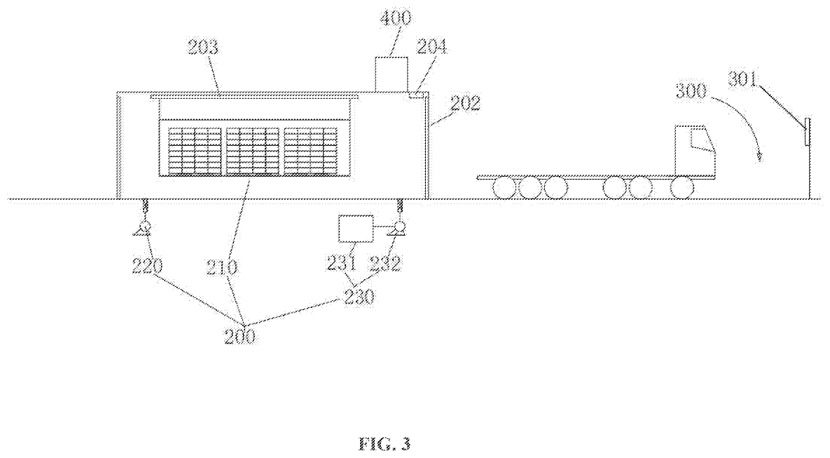

S9, starting an ozone supply device to fill ozone into the disinfection and sterilization bin, so that cold-chain materials in the refrigerated container, cold-chain material packages and gaps of the refrigerated container are filled with ozone, as shown in FIG. 3;

S8, enabling the pressure of the injected ozone to be 0.8 to 1.2 atmospheric pressures;

S11, maintaining the pressure of the ozone in the disinfection find sterilization bin for 1-3600 seconds; closing the door of the refrigeration container and/or the ventilation opening of the refrigeration container;

S12, controlling to open the disinfection and sterilization bin outlet by a central controller, and meanwhile, sending an instruction to prompt the transportation vehicle to return to the disinfection and sterilization bin by the second prompter;

S13, enabling the transportation vehicle to stop stably, and controlling the detaching and hanging device to operate by the central controller, so that the container descends to the transportation vehicle; and

S14, sending an instruction to the second prompter to prompt the refrigerated container transportation vehicle to drive out of the disinfection and sterilization bin by the central controller.

In an embodiment, in step S14, before the refrigerated container transportation vehicle is driven out of the disinfection and sterilization bin, a coding device is used for coding and recording on the refrigerated container transportation vehicle.

In conclusion, the disinfection and sterilization apparatus and the disinfection and sterilization method can effectively kill novel coronavirus in cold-chain food, cold-chain food packages and cold-chain food packaging boxes; and the container, the articles in the container and packaging boxes of the articles in the container are hung in the disinfection and sterilization bin through the detaching and hanging device, ozone can be easily diffused in the refrigerated container, and viruses on the surfaces of the packaging boxes of the articles in the container are efficiently eliminated and killed. According to the disinfection and sterilization apparatus and the disinfection and sterilization method, the disinfected and sterilized vehicles are coded, so that the traceability is realized. After the ozone used in the disinfection and sterilization apparatus is reacted, high-concentration oxygen can keep the freshness of cold-chain materials.

Obviously, the embodiments of the present disclosure are merely illustrative of the present disclosure as examples and are not intended to be limitation of embodiments of the present disclosure. For those of ordinary skill in the art, other variations or modifications in different forms may be made on the basis of the above description. All embodiments are impossible to be exhaustive herein. Obvious changes or variations which belong to the technical scheme of the present disclosure are still within the scope of protection of the present disclosure.

1. A disinfection and sterilization apparatus for a refrigerated container transportation system, comprising a to-be-treated waiting area, a disinfection and sterilization system, a treatment waiting area and a central control system, wherein
the to-be-treated waiting area, the disinfection and sterilization system and the treatment waiting area are sequentially arranged from left to right;
a first prompter is arranged in the to-be-treated waiting area;
the disinfection and sterilization system comprises a disinfection and sterilization bin, a vacuumizing device and an ozone supply device; a disinfection and sterilization bin inlet and a disinfection and sterilization bin outlet are respectively formed in the left side and the right side of the disinfection and sterilization bin; the disinfection and sterilization bin communicates with the vacuumizing device through a pipeline; the disinfection and sterilization bin communicates with the ozone supply device through a pipeline;
the disinfection and sterilization bin is provided with a detaching and hanging device for detaching or hanging a refrigerated container and a transportation vehicle; a second prompter is arranged in the treatment waiting area; and
the central control system is connected with the first prompter, the disinfection and sterilization bin inlet, the disinfection and sterilization bin outlet, the detaching and hanging device and the second prompter through data transmission lines, and the central control system is used for controlling the operation and the closing of the first prompter, the disinfection and sterilization bin inlet, the disinfection and sterilization bin outlet, the detaching and hanging device and the second prompter.

2. The disinfection and sterilization apparatus for a refrigerated container transportation system according to claim 1, wherein the disinfection and sterilization apparatus is further provided with a coding device; and the operation and the closing of the coding system are controlled by the central control system.

3. The disinfection and sterilization apparatus for a refrigerated container transportation system according to claim 1, wherein the disinfection and sterilization apparatus is further internally provided with a refrigerating device; and the operation and the closing of the refrigerating system are controlled by the central control system.

4. The disinfection and sterilization apparatus for a refrigerated container transportation system according to claim 1, wherein the vacuumizing device comprises a vacuumizing pump.

5. The disinfection and sterilization apparatus for a refrigerated container transportation system according to claim 1, wherein the ozone supply device comprises an ozone generator and an ozone pump.

6. The disinfection and sterilization apparatus for a refrigerated container transportation system according to claim 1, wherein the disinfection and sterilization bin inlet and the disinfection and sterilization bin outlet are the same port, and the to-be-treated waiting area and the treatment waiting area are the same area.

7. The disinfection and sterilization apparatus for a refrigerated container transportation system according to claim 1, wherein a vibration device is arranged on the detaching and hanging device, so that the detaching and hanging device has a vibration function.

8. A disinfection and sterilization method using the disinfection and sterilization apparatus according to claim 1, comprising the following steps:
S1, enabling a refrigerated container transportation vehicle to wait in a to-be-treated waiting area;
S2, sending an instruction to a first prompter to prompt the refrigerated container transportation vehicle to enter a disinfection and sterilization bin by a central control system;
S3, enabling the refrigerated container transportation vehicle to enter the disinfection and sterilization bin through the disinfection and sterilization bin inlet and stop stably;
S4, controlling the detaching and hanging device to operate by the central control system, so that the refrigerated container is lifted to be detached from the transportation vehicle;
S5, controlling a second prompter to send an instruction to prompt the transportation vehicle to leave the disinfection and sterilization bin by the central control system;
S6, opening a door of the refrigeration container and/or a ventilation opening of the refrigeration container to guarantee the vacuumizing effect and the ozone injection effect;
S7, closing the disinfection and sterilization bin inlet and the disinfection and sterilization bin outlet, so that the disinfection and sterilization bin is closed;
S8, starting a vacuumizing system to vacuumize the disinfection and sterilization bin;
S9, starting an ozone supply device to fill ozone into the disinfection and sterilization bin, so that cold-chain materials in the refrigerated container, cold-chain material packages and gaps of the refrigerated container are filled with ozone;
S8, enabling the pressure of the injected ozone to be 0.8 to 1.2 atmospheric pressures;
S11, maintaining the pressure of the ozone in the disinfection and sterilization bin for 1-3600 seconds; closing the door of the refrigeration container and/or the ventilation opening of the refrigeration container;
S12, controlling to open the disinfection and sterilization bin outlet by a central controller, and meanwhile, sending an instruction to prompt the transportation vehicle to return to the disinfection and sterilization bin by the second prompter;
S13, enabling the transportation vehicle to stop stably, and controlling the detaching and hanging device to operate by the central controller, so that the container descends to the transportation vehicle; and
S14, sending an instruction to the second prompter to prompt the refrigerated container transportation vehicle to drive out of the disinfection and sterilization bin by the central controller.

9. (canceled)

10. The disinfection and sterilization method according to claim 8, wherein the disinfection and sterilization apparatus is further provided with a coding device; and the operation and the closing of the coding system are controlled by the central control system.

11. The disinfection and sterilization method according to claim 8, wherein the disinfection and sterilization apparatus is further internally provided with a refrigerating device; and the operation and the closing of the refrigerating system are controlled by the central control system.

12. The disinfection and sterilization method according to claim 8, wherein the vacuumizing device comprises a vacuumizing pump.

13. The disinfection and sterilization method according to claim 8, wherein the ozone supply device comprises an ozone generator and an ozone pump.

14. The disinfection and sterilization method according to claim 8, wherein the disinfection and sterilization bin inlet and the disinfection and sterilization bin outlet are the same port, and the to-be-treated waiting area and the treatment waiting area are the same area.

15. The disinfection and sterilization method according to claim 8, wherein a vibration device is arranged on the detaching and hanging device, so that the detaching and hanging device has a vibration function.

16. The disinfection and sterilization method according to claim 8, wherein in step S14, before the refrigerated container transportation vehicle is driven out of the disinfection and sterilization bin, a coding device is used for coding and recording on the refrigerated container transportation vehicle.

17. The disinfection and sterilization method according to claim 10, wherein in step S14, before the refrigerated container transportation vehicle is driven out of the disinfection and sterilization bin, a coding device is used for coding and recording on the refrigerated container transportation vehicle.

18. disinfection and sterilization method according to claim 11, wherein in step S14, before the refrigerated container transportation vehicle is driven out of the disinfection and sterilization bin, a coding device is used for coding and recording on the refrigerated container transportation vehicle.

19. The disinfection and sterilization method according to claim 12, wherein in step S14, before the refrigerated container transportation vehicle is driven out of the disinfection and sterilization bin, a coding device is used for coding and recording on the refrigerated container transportation vehicle.

20. The disinfection and sterilization method according to claim 13, wherein in step S14, before the refrigerated container transportation vehicle is driven out of the disinfection and sterilization bin, a coding device is used for coding and recording on the refrigerated container transportation vehicle.

21. The disinfection and sterilization method according to claim 14, wherein in step S14, before the refrigerated container transportation vehicle is driven out of the disinfection and sterilization bin, a coding device is used for coding and recording on the refrigerated container transportation vehicle.
